(19) United States
(12) Patent Application Publication
(10) Pub. No.: US 2003/0235877 A1
(43) Pub. Date: Dec. 25, 2003

(54) Conjugates of haptens to beta-lactam derivatives and their use for detecting and/or quantifying haptens in solution and device for implementation thereof

(75) Inventors: Michel Kohl, Liege (BE); Roger Renotte, Oreye (BE); Guy Sarlet, Seraing (BE); Robert Lejeune, Heusy (BE); Benoit Granier, Neupre (BE)

Correspondence Address: MERCHANT & GOULD PC, P.O. BOX 2903, MINNEAPOLIS, MN, 55402-0903, US

(73) Assignee: La Region Wallone, Brussels (BE)

(21) Appl. No.: 10/269,673

(22) Filed: Oct. 10, 2002

Related U.S. Application Data
(63) Continuation of application No. 10/075,648, filed on Feb. 13, 2002.
Application No. 10/075,648 is a continuation of application No. 09/915,211.
Application No. 09/915,211 is a continuation-in-part of application No. 09/171,819, filed on Jun. 11, 1999, now Pat. No. 6,436,649.
Application No. 09/171,819 is a 371 of international application No. PCT/BE97/00052, filed on Apr. 30, 1997.

(30) Foreign Application Priority Data
Apr. 30, 1996 (BE) 96/00384

(51) Int. Cl. G01N 33/53; G01N 33/537; G01N 33/543; C12N 9/80

(52) U.S. Cl. 435/7.92; 435/228

(57) ABSTRACT

The present invention is related to a conjugate of a hapten to a natural or synthetic β-lactam derivative, comprising at least a side chain, wherein the side chain of the β-lactam derivative is at least partially constitutive of the conjugating arm.

The invention relates also to a method for the immunoassay of the hapten involving said β-lactam derivative—hapten conjugate as an inhibitor for a β-lactamase or a penicillin detector capable of specific recognition of the β-lactamic moiety of said conjugate.

### FIELD OF THE INVENTION

The present invention describes the synthesis β-lactam of conjugates of haptens to β-lactam derivatives and relates to their use for detecting and/or quantifying haptens in solution as well as to the device for detection and/or quantification, in particular the kit allowing the screening and/or the assay, of those haptens.

### TECHNOLOGICAL BACKGROUND FORMING THE BASIS OF THE INVENTION AND STATE OF THE ART

During the past few years, major advances in the field of biotechnology have made it possible to discover many low molecular weight molecules that play important roles in human and animals. Such molecules may be naturally occurring hormones but also active components extracted from plants or chemically synthesized components. These are active in situ at very low concentrations so that their monitoring is very difficult and expensive using conventional analytical techniques.

To facilitate the determination of these low molecular weight molecules—also named haptens because they are not immunogenic without prior conjugation to immunogenic proteins—in the body fluids, attempts have been made to develop immunoassays, especially enzyme immunoassays. In these enzyme immunoassays, said haptens, conjugated to different compounds such as antibodies, enzymes, enzyme substrates, enzyme co-factors, enzyme sub-units and the like, compete with corresponding free haptens for limited binding sites of antibodies to give competitive immunoassays.

Examples of enzyme immunoassays using hapten conjugates to enzymes, substrates and co-factors as well as co-immobilization of haptens and enzymes in dextran cavities are described in a book dealing with alternative immunoassays (Voller A. and Bidwell D. W., in Alternative Immunoassays, Wiley, Chichester, 1986, p. 77-86) while immunoassays involving hapten conjugates to enzyme sub-units are described by Henderson et al., Clin. Chem., 32, 9, 1637-1641 (1986).

Inhibitors are also involved in immunoassays. Patent application EP-A-017 648 describes an immunoassay wherein a peptidic inhibitor conjugated to a steroid or an aflatoxin delays the clotting of milk. Patent application EP-A-0 532 187 describes another immunoassay wherein an inhibitor conjugated to a high molecular weight agonist constituting a binding pair is only capable of modulating the activity of an enzyme conjugated to another agonist constituting a second binding pair after a foreign reaction has occurred allowing a close proximity between the inhibitor and the enzyme. It will be observed that the complex involving the two binding pairs is a big complex and that the inhibitor conjugating arm is very long ensuring a sufficient inhibitor mobility for accessing to the enzyme binding site.

Although β-lactamases are high turn-over enzymes easily detectable at very low concentrations, they are rarely proposed as labels in immunoassays. Nevertheless, a paper deals with the use of a β-lactamase labelled antibody for the detection of steroids in bovine urine. The β-lactamase activity is revealed owing to the benzylpenicillin-starch-iodine reaction. The iodine-based system proposed in this paper has the disadvantage that iodine is lost by sublimation. This therefore constitutes a first source of instability. Moreover, starch paste is easily degraded by bacteria or fungi, which constitutes another source of instability.

Patent application FR-A-2 339 172 describes a reactive system for determining if uric acid or another reagent oxidizable by iodine exists in a liquid in a proportion greater than a predetermined quantity in an alkaline medium, said reagent comprising a water-activable iodine generator capable of liberating in situ an appropriate quantity of free iodine with an indicator to detect the presence of iodine. The iodine generator has the disadvantage that the iodide is oxidizable in the air and that the reagents have to be stocked in the dry state.

Finally, to date, conjugates of haptens to β-lactam derivatives have never been proposed for use in immunoassays and related techniques. Stable iodine-based systems for detecting β-lactamases have not been developed any further.

### AIMS OF THE INVENTION

The present invention aims to prepare hapten conjugates to β-lactam derivatives which are recognized by β-lactamases or related penicillin detectors (penicillin binding proteins).

A complementary goal of the invention is to design immunoassays involving said conjugates in association with the penicillin detector or a mixture of penicillin detectors selected for either adapting the sensitivity or enlarging the concentration range.

Another purpose of the invention is to detect β-lactamases using a stable iodine generator allowing for high sensitivity detection of these enzymes in aqueous medium. In particular, it is sought to obtain a method and a device which make it possible to detect haptens present in a human or animal physiological fluid, at concentrations of the order of 100 pM or higher, or even at concentrations of the order of 10 pM or higher.

A specific aim of the present invention is seeking to optimize the sensitivity of said method and device by reducing the “background noise” observed in the devices and methods of the state of the art through covalent binding of inhibitors to their specific detectors.

### DETAILED DESCRIPTION OF THE INVENTION

The present invention relates to synthesis procedures allowing for the conjugation of haptens to β-lactam derivatives. According to the invention, the conjugation is preferably made through ester, amide or ether linkages, the conjugating arm being each time it is possible totally or partially constitutive of the β-lactam derivative.

Advantageously, the conjugating arm is bound to the amine group located on the β-lactam nucleus (or ring).

For the person skilled in the art, it is understood that the side chain of a β-lactam derivative is located on the amine group of the β-lactam nucleus. Thus, preferably according to the invention, the side chain belongs to the conjugating arm.

According to the invention, the part of the conjugating arm (i.e. the arm skeleton) located between the hapten moiety and and the side chain end of the β-lactam derivative comprises between 0 and 10 atoms, preferably between 0 and 4 atoms, including possibly substituted carbon atoms as well as one to several heteroatoms. The heteroatoms can be O, S or N. For example, in the case of a steroid, the side chain oxygen atom located on the carbon 17 is considered to be integral of the steroid and is not counted.

The particular mode of conjugation claimed in the present invention guarantees to maintain kinetic parameters (Kₘ, Kᵢ, k_{cat} and Kₐ=k2/K, where Kₘ is the enzyme Michaelis-Menten constant, Kᵢ is the inhibitor-enzyme complex dissociation constant, k_{cat} the catalytic constant and Kₐ the apparent second-order rate constant for acyl enzyme formation respectively) toward β-lactamases and penicillin detectors, which are essentially the same as those of common penicillins.

According to the invention, the haptens are preferably steroids, drugs and drugs of abuse while the β-lactam derivatives have a nucleus consisting of a 2-azetidinone (beta-lactam) ring fused preferably to either a thiazolidine or a dihydro-1,3-thiazine ring. In the first case, the compounds are usually referred to generically as penicillins, whereas, in the second case, the compounds are referred to as cephalosporins.

The present invention also relates to a method involving the conjugates described above for detecting and/or quantifying a hapten in solution, in which:

a known quantity of an inhibitor-hapten conjugate is added to the solution containing the hapten to be detected and/or to be quantified;

a quantity of anti-hapten antibody corresponding to the quantity of the inhibitor-hapten conjugate is added to the solution;

a β-lactamase, preferably of class C, or a related penicillin detector, having a specific site for the hapten and the inhibitor-hapten conjugate entering into competition on said specific site eliciting a modulation of the enzyme or the penicillin detector response, is added to the solution, the response being measured by the detection and/or the quantification of a product which is detectable and/or quantifiable, preferably by visible UV radiation measurement.

Prior to the above-mentioned operations, substances may be optionally added to the solution containing the hapten to be assayed in order to remove possible interference such as agents for protecting the enzyme and the penicillin detectors, agents for protecting the enzyme substrates, agents for protecting the hapten-inhibitor conjugate or decontaminating agents and the like.

Some or all of the above-mentioned operations may be combined, that is to say may be carried out simultaneously, and/or the indicated order of the operations can be modified. It is therefore solely for clarity in the description that the different operations prior to the addition of an enzyme or a penicillin detector to the solution are presented in succession as separate steps.

Consequently, in the absence of free hapten, all the inhibitor-hapten conjugate molecules will have bound an antibody and will consequently be inactive. The response of the enzyme or the penicillin detector will consequently be maximum. On the other hand, the higher the quantity of hapten to be assayed, the lower the quantity of inhibitor-hapten-antibody complex, which implies a substantial availability of the molecules of inhibitor-hapten conjugate. This will lead to a low response of the system.

The present invention also relates to a device for screening and/or quantifying a hapten in solution, comprising a β-lactamase or a penicillin detector, having a specific site for the hapten and the inhibitor-hapten conjugate entering into competition on said specific site eliciting a modulation of the enzyme or the penicillin detector response, the response being measured by the detection and/or the quantification of a product which is detectable and/or quantifiable, preferably by visible UV radiation measurement. Said device also comprises antibodies capable of binding said inhibitor-hapten conjugate.

The covalent link between the conjugates and the β-lactamase is not stable, while the conjugates undergo completely irreversible binding to penicillin detectors. This permits to obtain a better sensitivity.

In the method and the device according to the invention, the β-lactamase is advantageously chosen from the group consisting of β-lactamases obtained from Enterobacter cloacae Q908R and P99 and the β-lactamases obtained from Citrobacter freundii and Escherichia coli while the penicillin detector is a penicillin binding protein included in a kit advantageously chosen from the group consisting of Penzym® (DD-peptidase extracted from Actinomadura R39, UCB Bioproducts, Belgium), SNAP® Beta Lactam (Idexx, US), BetaSTAR® (UCB, Belgium), Parallux® Beta Lactam assay (Idexx, US), Charm Farm Test® (Charm Sciences Inc., US), Delvo-X-press®, Delvotest P® and Delvo® test SP (DSM, The Netherlands).

According to the invention, the enzyme substrate is preferably chosen from the group consisting of cephaloridine, nitrocefin, cephalothin, cephalexin, cephalosporin C, cephacetrile and cefazolin.

According to the invention, the inhibitor-hapten conjugate is preferably chosen from the group consisting of carbenicillin, oxacillin, cefuroxine, cefotaxime, methicillin, ampicillin, cloxacillin and benzylpenicillin conjugates.

The invention also relates to a method of detection by a color system using an iodine/starch paste system stabilized by addition of cadmium iodide.

Preferably, the demonstration of the reaction is detected by measuring the color observed in the visible region or, when the products are not colored, by an indicator system, in particular by coloring with iodine/starch.

The Applicant noticed that cadmium iodide is stable with respect to oxidation in the air and that the antibacterial properties of the cadmium ion stabilize starch paste to pollution by microorganisms.

A system of the type mentioned, based on the generation of iodine in a starch paste solution stabilized by addition of cadmium iodide, proved particularly favorable in the above-mentioned technique. Cadmium iodide, in the presence of DTPA and iodate, reacts in a medium of pH 2 to produce the reagent which can then be brought to the working pH. It should be noted that this type of reaction may also be suitable for other assays, such as the assay of cephalexin with iodine, as will be described in an exemplary embodiment below.

In the particular case of the detection and/or the quantification (assay) of the concentration of the product resulting from the activity of the label which was mentioned above, this starch/iodine color allows the detection and/or the assay of a hapten at low concentrations and in a range of colors which discriminates with respect to the general color of the medium.

In the method and the device according to the invention, the inhibitor-hapten conjugate is preferably chosen from the group consisting of carbenicillin, oxacillin, cefuroxine, cefotaxime, methicillin, ampicillin, cloxacillin and benzylpenicillin conjugates as well as any other conjugates with a β-lactam ring or a ring related to the β-lactam ring, or even any substance not necessarily possessing a β-lactam ring or a ring related to the β-lactam ring but exhibiting measurable kinetic parameters (Kₘ, Kᵢ, k_{cat} and Kₐ=k₂/K, where Kₘ is the enzyme Michaelis-Menten constant, Kᵢ is the inhibitor-enzyme complex dissociation constant, k_{cat} the catalytic constant and Kₐ the apparent second-order rate constant for acyl enzyme formation respectively) toward the above-mentioned group of β-lactamases and penicillin detectors and ensuring a modulation of the colored signal produced in the reaction of the β-lactamase or the penicillin detector.

Advantageously, the hapten to be assayed is a medicine active component, a hormone, an anabolic steroid or a drug which is preferably chosen from the group consisting of testosterone, estradiol, progesterone, aldosterone, cortisol, methadone, methylamphetamine, tetrahydrocannabinol, Δ⁴-androstenedione, morphine, DHEA sulfate, nandrolone, theophylline, cocaine and/or their hydrolysis derivatives.

### BRIEF DESCRIPTION OF THE DRAWINGS

FIG. 1 describes the synthesis of nandrolone carbenicillinate (conjugate 1).

FIG. 2 describes the synthesis of progesterone benzylpenicillinate (conjugate 2).

FIG. 3 describes the synthesis of cocaine carbenicillinate (conjugate 3).

FIG. 4 describes the synthesis of precursors (phenyloxazoles) for steroid oxacillinates.

FIG. 5 describes the synthesis of precursors 5, 6 and 7.

FIG. 6 describes the synthesis of precursors 8, 9, 10, 11 and 12.

FIG. 7 describes the synthesis of precursors 13 and 14.

FIG. 8 describes the formation of oxacillin side chains by reacting precursors 1 to 5 with precursors 6 to 14 and chains 4 to 23.

FIG. 9 describes the synthesis of chains 24-32, the coupling of chains 4-32 to 6-APA and the deprotection of estradiol-containing conjugates.

FIG. 10 shows the variation of [(V_{ab}/V_{dos})−1] as a function of the quantity of nandrolone.

FIG. 11 shows a progesterone assay using a Beta-Star® kit penicillin detector.

### DESCRIPTION OF A PREFERRED EMBODIMENT OF THE INVENTION

1. Synthesis Routes of the Conjugate

In order to reduce at a minimum the length of the conjugating arm between the hapten and the β-lactam derivative, the conjugating arm is constructed from the side chain of the β-lactam derivative: the native side chain containing a carboxyl group available for esterification or a closely related analogue substituted by a carboxyl group is activated to form the corresponding monoacyl chloride. The latter is reacted to hydroxyl bearing haptens or to closely related hapten analogues bearing such hydroxyl groups forming precursors in which the components are linked through ester functions. The remaining carboxyl, activated using carbonyl diimidazole is reacted with 6-aminopenicillinic acid forming the expected conjugate.

2. Selection of the Penicillin Detector

Depending on the sensitivity and the range expected for the assay, a detection system based on either a β-lactamase enzyme (Class C β-lactamase as that extracted from Enterobacter cloacae P99) or a penicillin detector (Penzym® or BetaSTAR®) is chosen.

3. Choice of the Conjugate

This choice greatly depends on the penicillin detector. Benzylpenicillin conjugates work efficiently with BlaR or R39 while β-lactamases are only significantly inhibited by conjugates including carbenicillin or oxacillin moieties. Using β-lactamases, higher sensitivities are achieved with oxacillin conjugates. The conjugate will thus be selected according to the normal concentration of the analyte in the considered medium and the kinetic parameters for the selected enzyme or detector.

4. Detection

β-lactamases are detected through the appearance of degradation colored products of a reporter substrate characterized by its Kₘ and k_{cat}. The corresponding high values for nitrocefin and cephalexin make them suited candidates as reporter substrates. The penicillin detectors having lost any enzymatic activity are detected through the associated colored particles or through the colored compounds generated by an associated enzyme label.

Synthesis of the Conjugates

1. Synthesis of Nandrolone Carbenicillinate (Conjugate 1)

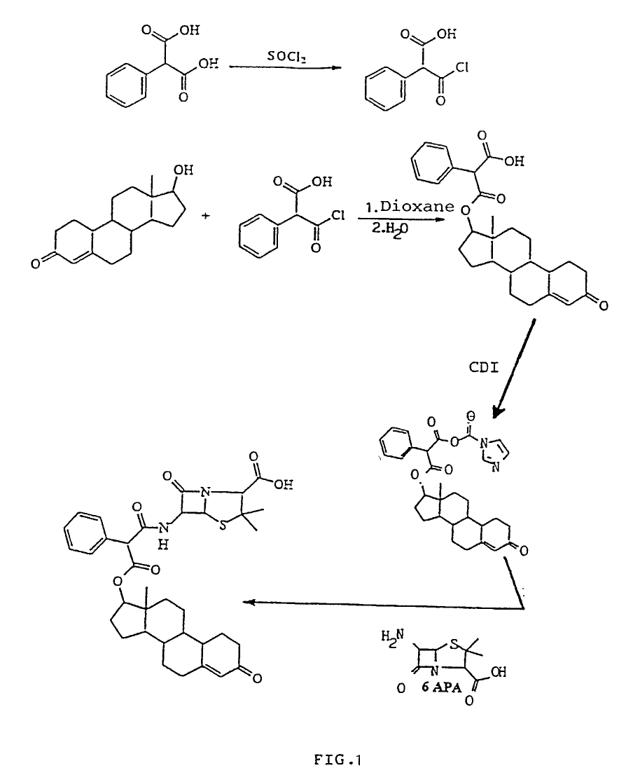

Phenylmalonic acid is converted to an acid monochloride by incubation at 35° C. in a dry dioxane-ether mixture with 1.2 equivalents of thionyl chloride for 3 hours. The solvents are evaporated and the oily residue, taken up in a dioxane-ether mixture, is mixed with an ice-cold solution of nandrolone in dioxane. The mixture in which the steroid is deficient is incubated at room temperature for 12 hours. The product is extracted with an aqueous sodium bicarbonate solution, and then after acidification of the solution to pH 2, re-extracted with chloroform. The steroid hemi-phenylmalonate is obtained by evaporation of the chloroform (see FIG. 1).

The coupling of the nandrolone hemi-phenylmalonate to 6-APA is carried out conventionally after activation with carbonyldiimidazole (CDI). The purification of the conjugate is obtained by extraction in ether in a medium of pH 2 and 8.5 before final recrystallization from a toluene-white petrolatum mixture (see Table 1).

2. Synthesis of Progesterone Benzylpenicillinate (Conjugate 2)

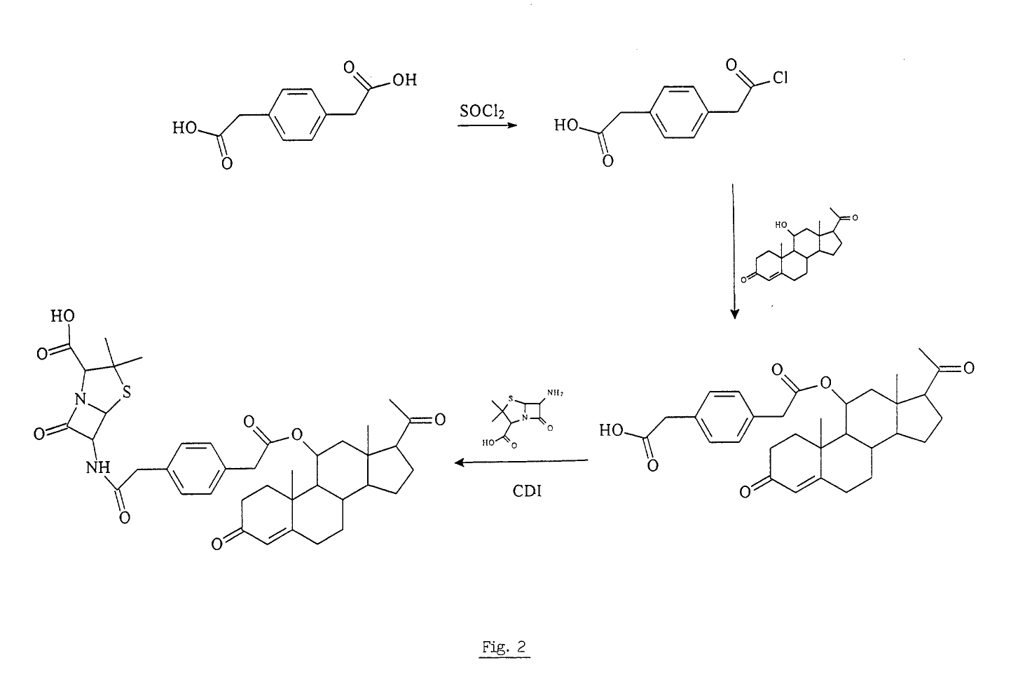

Phenyldiacetic acid is converted to an acid monochloride by incubation at 35° C. in a dry dioxane-ether mixture with 1.2 equivalents of thionyl chloride for 3 hours. The solvents are evaporated and the oily residue, taken up in a dioxane-ether mixture, is mixed with an ice-cold solution of 11α-hydroxyprogesterone in dioxane. The mixture in which the steroid is deficient is incubated at room temperature for 12 hours. The product is extracted with an aqueous sodium bicarbonate solution, and then after acidification of the solution to pH 2, re-extracted with chloroform. The progesterone hemi-phenyldiacetate is obtained by evaporation of the chloroform (see FIG. 2).

The coupling of the progesterone hemi-phenyldiacetate to 6-APA is carried out conventionally after activation with carbonyldiimidazole (CDI). The purification of the conjugate is obtained by extraction in ether in a medium of pH 2 and 8.5 before final recrystallization from a toluene-white petrolatum mixture (see Table 1).

3. Synthesis of Cocaine Carbenicillinate (Conjugate 3)

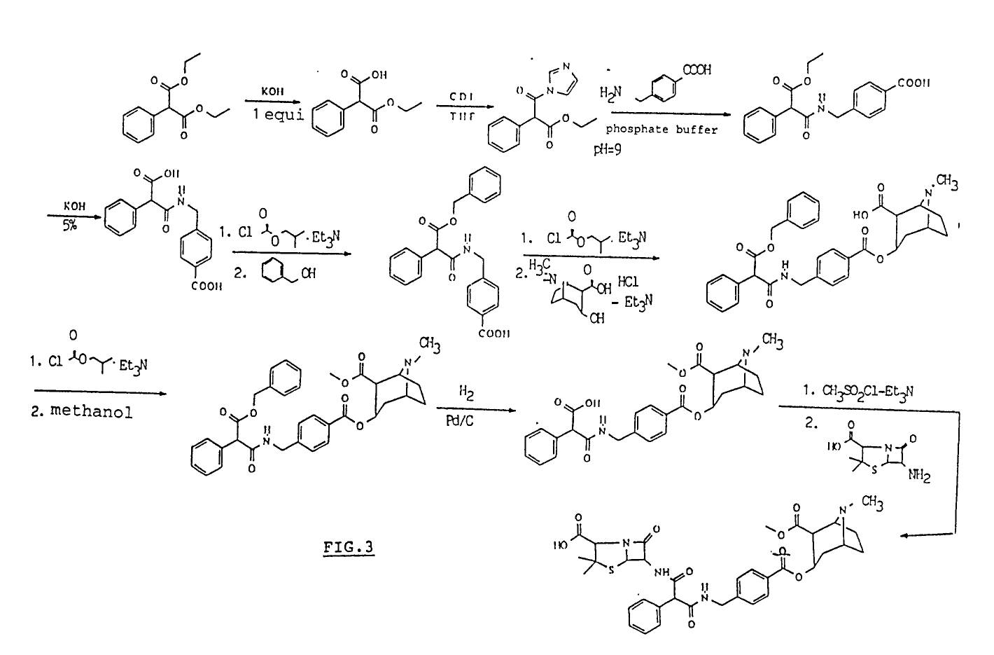

Diethyl phenylmalonate is hydrolysed to a monoester by the action of one equivalent of KOH in an aqueous-alcoholic medium. The monoester is then conjugated with p-aminomethylbenzoic acid by a conventional procedure involving carboxyl diimidazole as activating agent. The residual ethyl ester is replaced with a corresponding benzyl ester by hydrolysis with 5% KOH followed by esterification with benzyl alcohol using the method of mixed anhydrides with isobutyl chloroformate in the presence of one equivalent of triethylamine. The remaining carboxyl functional group is conventionally activated with isobutyl chloroformate and then coupled to ecgonine. The carboxylic acid of the benzoylecgonine portion of the conjugate is converted by methyl ester by the same technique applied in the presence of methanol. The benzyl functional group is removed by hydrogenolysis in the presence of palladium on carbon at a hydrogen pressure of two bar. The acid generated is used for the final coupling to 6-APA using prior activation with methanesulfonyl chloride according to the method of Brown et al., Chem. Soc. Perkins Trans No.1, p. 881, 1991 (see FIG. 3). The purification of this compound is obtained by extraction in ether in a medium of pH 2 and 8.5 before final recrystallization from the toluene-white petrolatum mixture (see Table 1).

4. Synthesis of Various Steroid Oxacillinates

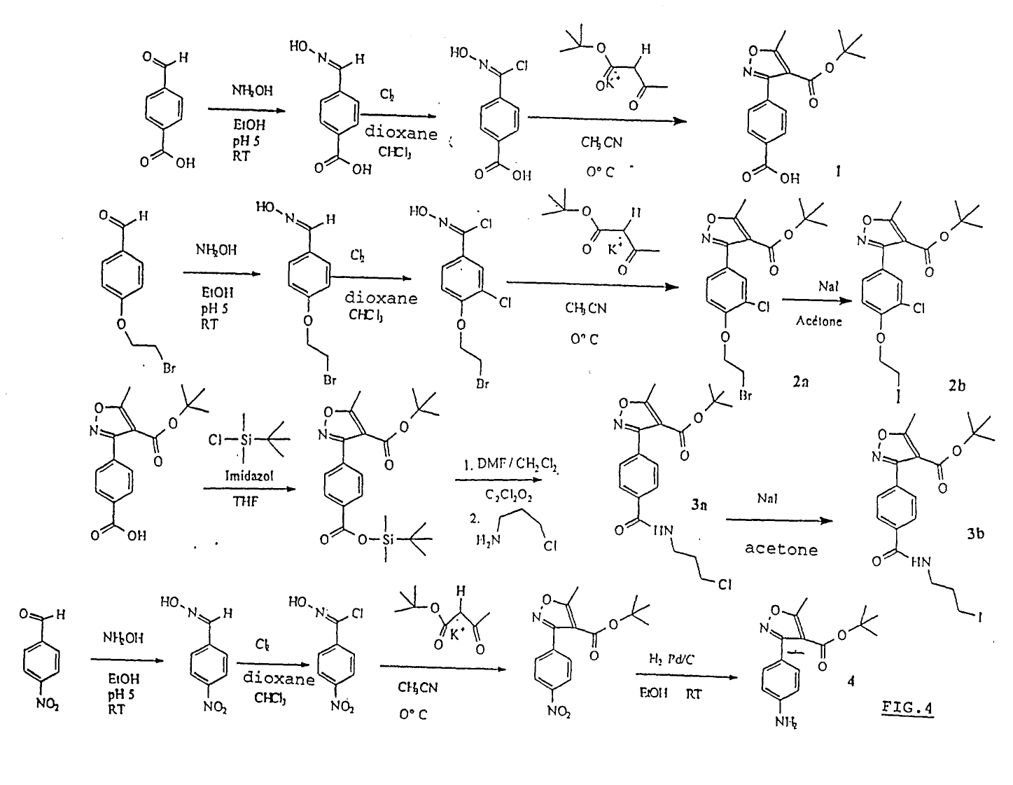

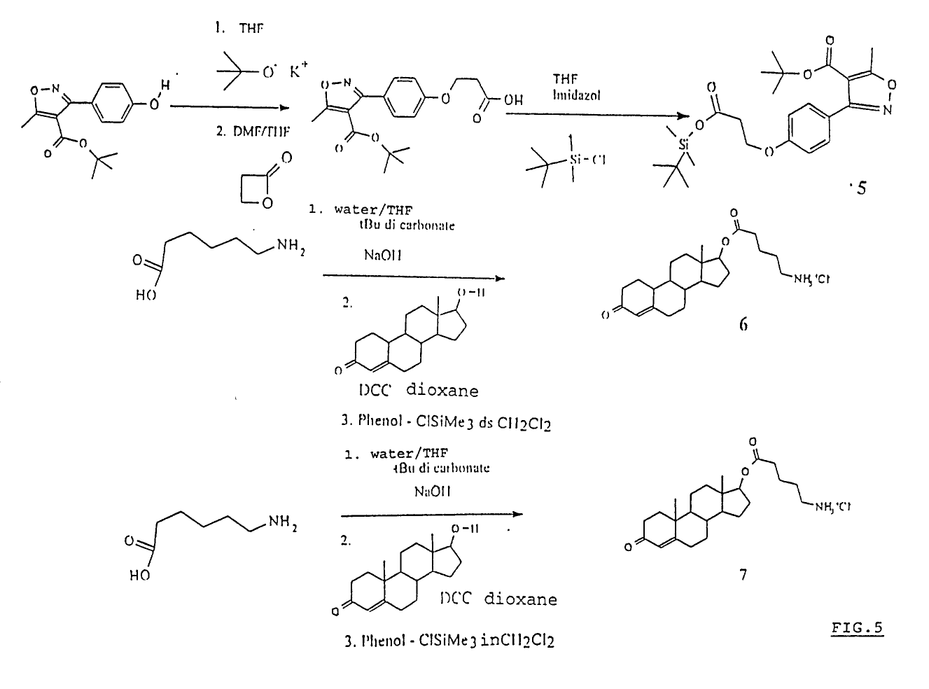

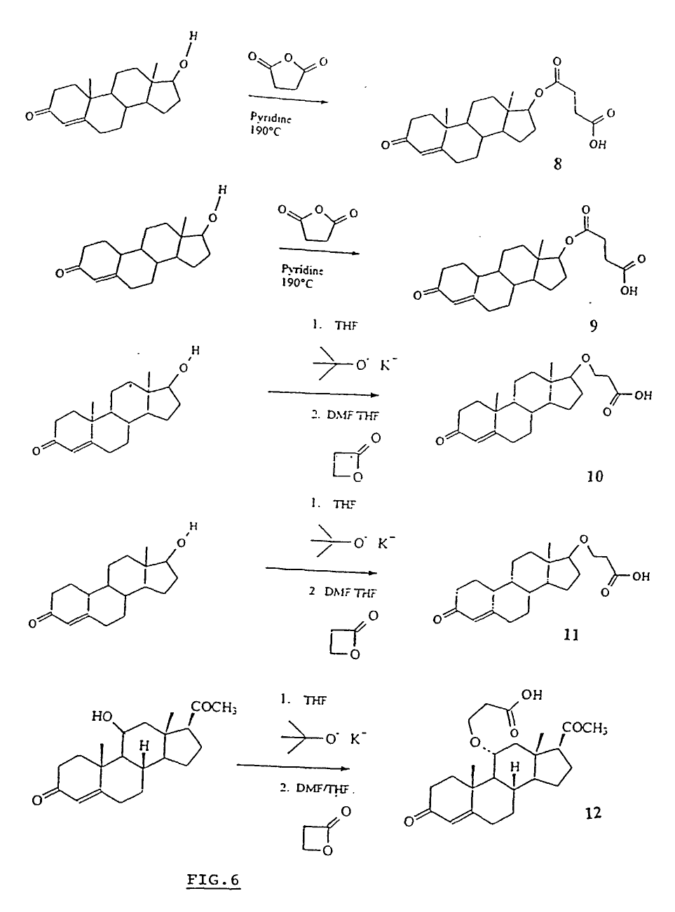

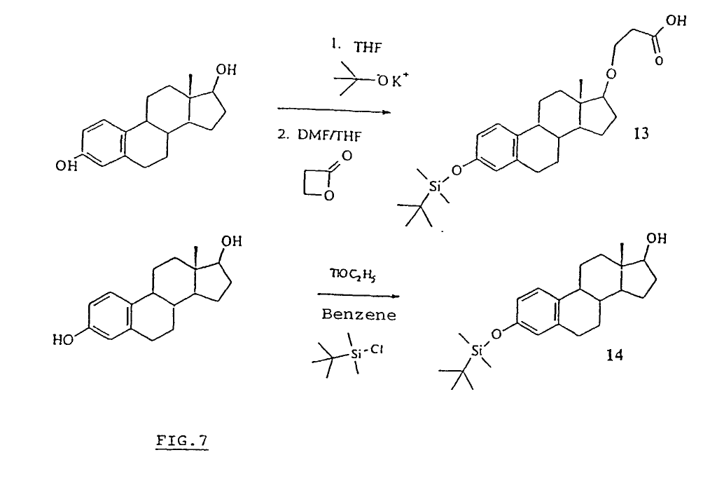

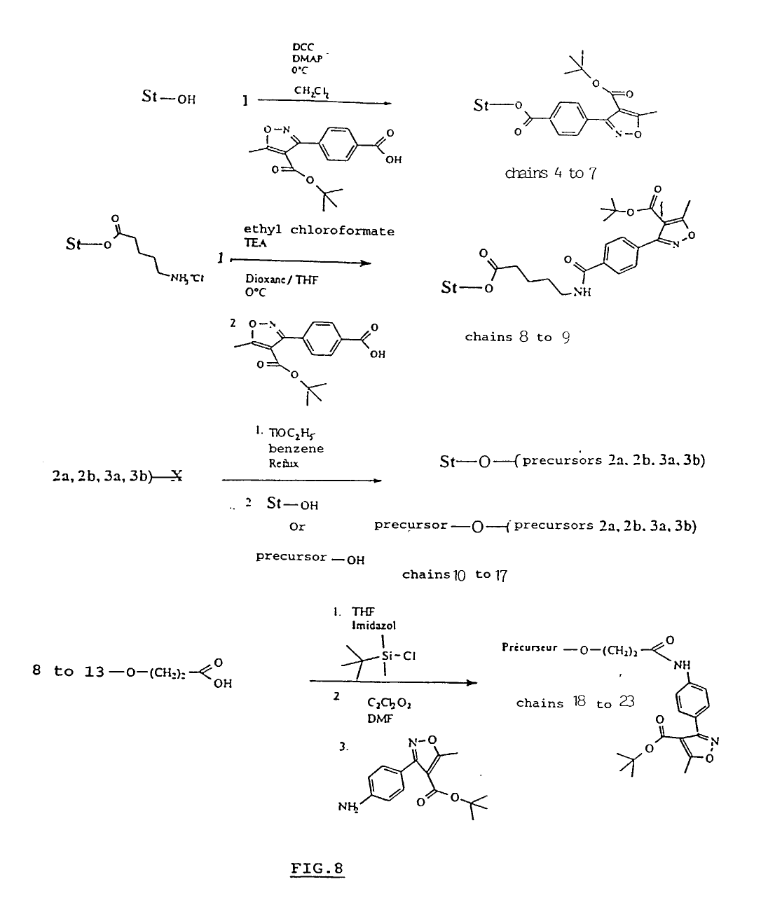

The procedure followed in order to obtain the target molecules consists in synthesizing different precursors comprising the steroid portion possibly modified by the addition of a conjugating arm and the oxacillin side chain appropriately modified in order to allow coupling to the steroid or the modified steroid through either ester or ether or amide bonds. Next, an amide is formed by coupling the precursor to 6-aminopenicillanic acid (6-APA) in order to obtain the final molecule. The general method of synthesis presented below is summarized in FIGS. 4 to 9.

4.1. Synthesis of the Precursors (Phenylisoxazoles)

4.1.1. Synthesis of tert-butyl 3-(4-carboxyphenyl)-5-methylisoxazole-4-carboxylate (precursor 1)

One equivalent of 4-carboxybenzaldehyde (I) is reacted with two equivalents of hydroxylamine hydrochloride in a water/methanol mixture adjusted to pH 4.5 with NaOH. After two hours of reaction, the solution is concentrated under vacuum until precipitation of the product (II) is obtained. The precipitate is filtered, washed with ice-cold water and dissolved in a NaHCO₃ buffer at pH 8. The product solution is purified using charcoal, filtered and acidified with HCl. The product II which precipitates is isolated, washed with H₂O and dried under vacuum.

The product II is dissolved in a dioxane/CHCl₃ mixture. The solution, cooled in an acetone/dry ice mixture, is then saturated with chlorine. The chlorinated solution is then heated gradually up to room temperature. After complete reaction, the solution is evaporated under vacuum. The product obtained (III) is dissolved in ethanol and precipitated by addition of white petrolatum.

One equivalent of product III is dissolved in a methanol/acetonitrile mixture. After cooling to 0° C., two equivalents of Na tert-butyl acetoacetate (IV) are slowly added to the solution of product III. At the end of the reaction, the solution is supplemented with water acidified with acetic acid. The product is extracted from the solution with chloroform. The chloroformic phase, washed with water, is dried over sodium sulfate. It is then evaporated until the product (V) is concentrated and precipitated by addition of white petrolatum giving the precursor 1.

4.1.2. Synthesis of tert-butyl 3-[4-(2-bromoethoxy)-3-chlorophenyl]-5-methylisoxazole-4-carboxylate and tert-butyl 3-[4-(2-iodoethoxy)-3-chlorophenyl]-5-methyl-isoxazole-4-carboxylate (Precursors 2a and 2b)

The synthesis is based on the preparation of the preceding product, the starting material being bromoethoxybenzaldehyde. The bromoethoxybenzaldehyde is converted to an oxime by the action of hydroxylamine hydrochloride in an aqueous-alcoholic medium whose pH is kept at 5 by controlled addition of NaOH. The action of the chlorine at saturation in chloroform for 2 hours converts the oxime to a chloroxime with subsequent substitution of the phenyl ring with a chlorine atom at the ortho position of the ether. The product is purified twice by chromatography on a silica column, the mobile phase consisting of toluene, ethyl acetate and acetic acid (6;1;0.1) in order to eliminate the unstable compounds which form during the reaction. The stable compound obtained (Table 9) is then reacted with 1 equivalent of potassium salt of tert-butyl acetoacetate. The addition compound cyclizes during the reaction in order to give tert-butyl 3-[4-(2-bromoethoxy)-3-chlorophenyl]-5-methylisoxazole-4-carboxylate (precursor 2a), which can be converted to a corresponding iodinated derivative by reaction with NaI in an acetonic medium (precursor 2b).

4.1.3. Synthesis of tert-butyl 3-[N-(3-chloropropyl)benzamid-4-yl]-5-methylisoxazole-4-carboxylate and tert-butyl 3-[N-(3-iodopropyl)benzamid-4-yl]-5-methylisoxazole-4-carboxylate (Precursors 3a and 3b)

The tert-butyl 3-(4-carboxyphenyl)-5-methyl-isoxazole-4-carboxylate is protected by forming a tert-butyldimethylsilyl derivative on the carboxyl functional group by reacting with tert-butyldimethylsilyl chloride in the presence of imidazole. The protected derivative is converted to an acid chloride by the action of oxalyl chloride in a mixture of dimethylformamide and dichloromethane. An amide is then formed by reacting the acid chloride with chloropropylamine introduced in the hydrochloride form into the medium supplemented with an excess of triethylamine. The chlorinated derivative (precursor 3a) thus obtained may be converted to the corresponding iodinated derivative (precursor 3b) by reacting with NaI in acetone.

4.1.4. Synthesis of tert-butyl 3-(4-aminophenyl)-5-methylisoxazole-4-carboxylate (Precursor 4)

The 4-nitrobenzaldehyde is converted to an oxime by reacting with hydroxylamine in an aqueous-alcoholic medium at pH 5 (maintenance of the pH by addition of KOH), then the chlorooxime is formed by chlorination with the aid of gaseous chlorine in chloroform. The reaction of the derivative obtained with the potassium salt of tert-butyl acetoacetate gives tert-butyl 3-(4-nitrophenyl)-5-methylisoxazole-4-carboxylate, which is reduced with the aid of hydrogen (2 bar) catalyzed by palladium on carbon. The amine-containing derivative is isolated in hydrochloride form in ethyl acetate.

4.1.5. Synthesis of tert-butyl 3-[4-(2-carboxyethoxy)phenyl]-5-methylisoxazole-4-carboxylate Protected by a tert-butyldimethylsilyl Ester (Precursor 5)

Para-Hydroxybenzaldehyde is reacted with propiolactone in the presence of potassium tert-butoxide in a mixture of dimethylformamide and acetonitrile. The derivative formed is converted to an oxime as described above for the related compounds and protected by reacting with 1 equivalent of tert-butyl dimethylsilyl chloride for 2 hours in tetrahydrofuran supplemented with imidazole. The action of chlorine on the product gives the chloroxime which is converted to an isoxazole by the potassium salt of tert-butyl acetoacetate according to the method already described.

4.2. Preparation of Steroids for Coupling to Phenylisoxazoles

Nandrolone and testosterone were used without modification or after addition of a 5-aminovaleric or 3-hydroxypropionic arm.

6.4.2.1. Aminovaleric derivatives of nandrolone (precursor 6) or of testosterone (Precursor 7)

The 5-aminovaleric acid is protected with a Boc group by reaction of di-tert-butyl dicarbonate in a water-tetrahydrofuran mixture in the presence of NaOH. The derivative obtained is put in reaction with a deficient quantity of testosterone or nandrolone in the presence of a slight excess of dicyclohexylcarbodiimide and of dimethylaminopyridine. The reaction is carried out in a mixture of dioxane and tetrahydrofuran at a temperature of 0° C. The protection of the amine group is removed by reaction of 4 equivalents of phenol and of 1 equivalent of tetramethylsilyl chloride in a mixture of dichloromethane and ethyl acetate. The product formed is isolated from ether.

4.2.2. Hemisuccinates of Testosterone and of Nandrolone (Precursors 8 and 9)

The steroid is reacted with 1 equivalent of succinic anhydride in a solution of pyridine heated in an autoclave at a temperature of 120° C.

4.2.3. Addition of a Propionic Arm to Testosterone and to Nandrolone (Precursors 10 and 11) as well as to Progesterone (Precursor 12)

The potassium salt of the steroid (11α-hydroxyprogesterone, nandrolone, and testosterone) is formed by reacting 1 equivalent of steroid with 1 equivalent of potassium tert-butoxide in a tetrahydrofuran medium. After 15 minutes, the medium is diluted with three volumes of dimethylformamide and 1 equivalent of β-propiolactone is added.

4.2.4. Preparation of 3-tert-butyldimethylsilylestradiol (Precursor 14) and Addition of a Propionic Arm to Protected Estradiol (Precursor 13)

Estradiol and thallium ethoxide, in equivalent quantities, are heated in benzene, continuously removing the alcohol which is liberated by azeotropic distillation. When the reaction is complete, 1.1 equivalent of tert-butyldimethylsilyl chloride is added and the reaction is continued until the substitution of the phenol is complete (precursor 14). The potassium salt of the protected steroid is formed and it is substituted with the propionic chain as described above for precursors 10 to 12 (precursor 13).

4.3. Formation of Oxacillin Side Chains by Reacting Precursors 1 to 5 with Precursors 6 to 14

Precursor 1 is activated in solution in dichloromethane supplemented with a trace of dimethylformamide in the presence of 5 mg % of dimethylaminopyridine and 1 equivalent of either precursor 14, or of nandrolone or of testosterone, or of 11-α-hydroxyprogesterone and of a slight excess of dicyclohexylcarbodiimide gradually added to the solution cooled to 0° C. After removal of the dicyclohexylurea, the product is isolated from petroleum ether. Protection of the 4-isoxazolecarboxylic acid is removed by the action of trifluoroacetic acid in a solution of nitromethane heated under reflux for 40 minutes. The product is purified by chromatography on a silica column eluted with a toluene, ethyl acetate and acetic acid 2;1;0.1 mobile phase. Chains 4, 5, 6 and 7 result from this preparation.

Precursor 1 is activated with isobutyl chloroformate (1.1 equivalent) in dioxane containing a trace of dimethylformamide at a temperature of 0° C. in the presence of a slight excess of triethylamine. A solution, in dimethylformamide, of one of precursors 6 or 7 is added to this solution and the reaction is allowed to proceed in the presence of an excess of triethylamine in order to obtain chains 8 and 9 after deprotection as above.

Any one of precursors 14, nandrolone, testosterone or 11-α-hydroxyprogesterone and thallium ethoxide placed in equivalent quantities in benzene are heated while continuously removing the alcohol which is liberated by distillation of the benzene. When the reaction is complete, 1 equivalent of either precursor 2a or 2b depending on the reactivity, or of precursor 3a or 3b depending on the reactivity is added, and the reaction is continued until complete substitution is obtained. Chains 10 to 17 are obtained by this route after purification on a silica column with, as mobile phase, a mixture of white petrolatum and of toluene having a composition suited to the lipophilicity of the product, and the deprotection is carried out as above.

Any one of precursors 8 to 13 is converted to a tert-butyldimethylsilyl ester by reaction with 1 equivalent of tert-butyldimethylsilyl chloride in the presence of imidazole in a medium consisting of tetrahydrofuran. This ester is reacted in dichloromethane in the presence of oxalyl chloride and catalytic quantities of dimethylformamide in order to form the corresponding acid chloride which is coupled to precursor 4 introduced into the reaction medium in an equivalent quantity. Chains 18 to 23 are produced in this manner after deprotection according to the methods described above. Precursor 5 is converted to an acid chloride in dichloromethane medium by 1 equivalent of oxalyl chloride in the presence of catalytic quantities of dimethylformamide, and reacted with any one of the following products: testosterone, nandrolone, precursor 6, precursor 7, 11-α-hydroxyprogesterone and precursor 14 introduced into the reaction medium in equivalent quantities. Chains 24 to 29 are obtained after deprotection according to the method already described.

Additional informations can be obtained in the article of Kohl M. and Lejeune R., in Steroids (in press).

4.4. Coupling of Chains 4 to 29 to 6-APA

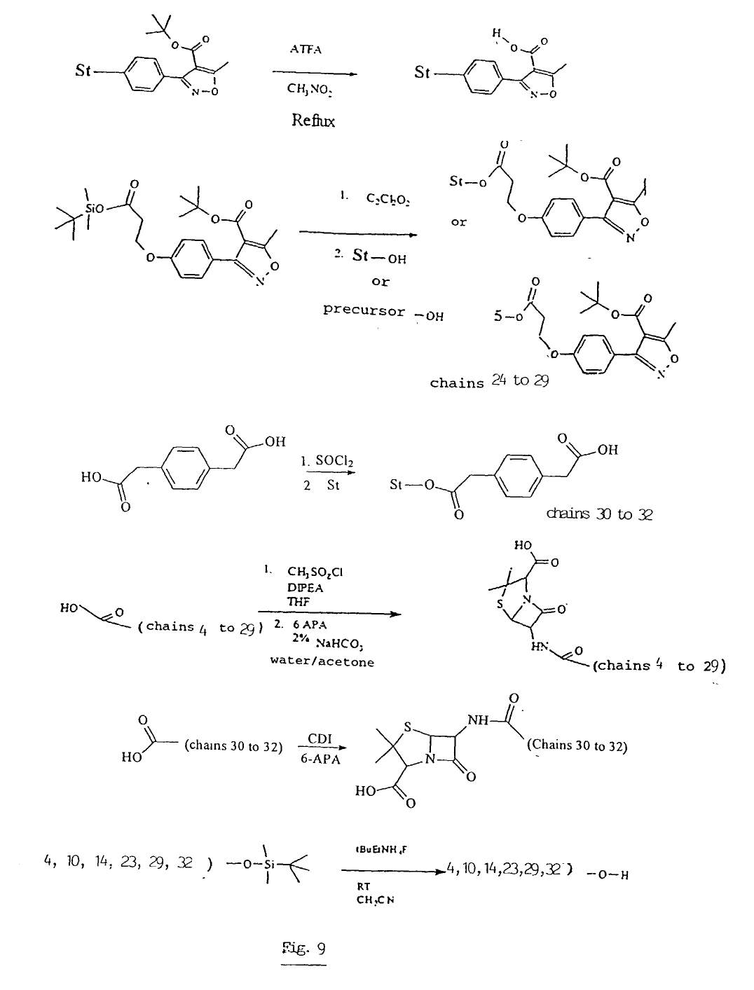

The carboxyl at position 4 of the isoxazole ring of the chain is activated with methanesulfonyl chloride (1 equivalent) in tetrahydrofuran medium cooled to −50° C. and containing 1 equivalent of diisopropylethylamine. After reaction and returning to room temperature, 1 equivalent of 6-aminopenicillanic acid, dissolved in an aqueous solution of bicarbonate at 2% and diluted with an equal volume of acetone is introduced into the medium. After removal of the solvents and purification by extraction with ether starting with solutions at pH 2 and pH 8.5, the conjugates 4 to 29 are isolated from white petrolatum (FIG. 9). However, the products 4, 10, 14, 23 and 29 must undergo deprotection of the phenol with the HF/KF mixture at pH 5 in a tetrahydrofuran medium (FIG. 9).

The characteristics of these conjugates are presented in Table 1.

5. Synthesis of Various Steroid Benzylpenicillinates

The procedure followed in order to obtain the target molecules consists in synthesizing precursors comprising the steroid portion and the benzylpenicillin side chain appropriately modified in order to allow coupling to the steroid through ester bonds. Next, an amide is formed by coupling the precursor to 6-APA in order to obtain the final molecule. The general method of synthesis presented below is summarized in FIG. 9.

5.1. Formation of Benzylpencillin Side Chains by Reacting Phenyldiacetic Acid with Nandrolone, Testosterone and Precursor 14

Phenyldiacetic acid is converted to an acid monochloride by incubation at 35° C. in a dry dioxane-ether mixture with 1.2 equivalents of thionyl chloride for 3 hours. The solvents are evaporated and the oily residue, taken up in a dioxane-ether mixture, is mixed with an ice-cold solution in dioxane of any one of the following products: nandrolone, testosterone or precursor 14. The mixture in which the steroid or the precursor is deficient is incubated at room temperature for 12 hours. The product is extracted with an aqueous sodium bicarbonate solution, and then after acidification of the solution to pH 2, re-extracted with chloroform. The side chains 30 to 32 are obtained by evaporation of the chloroform (FIG. 9).

5.2. Coupling of Chains 30 to 32 to 6-APA

The coupling of the chains 30 to 32 to 6-APA is carried out conventionally after activation with carbonyldiimidazole (CDI). The purification of the conjugate is obtained by extraction in ether in a medium of pH 2 and 8.5 before final recrystallization from a toluene-white petrolatum mixture giving the conjugates 30 to 32 (see Table 1). However, the product 32 must undergo deprotection of the phenol with the HF/KF mixture at pH 5 in a tetrahydrofuran medium. The characteristics of these conjugates are shown in Table 1.

Legend of Table 1.

^{Ω}(Column 2): FAB

^{#}(Column 2): electrospray

⁺(Column 2): positive (M+H)

⁻(Column 2): negative (M−H)

^{x}(Column 9): calculated using the β-lactamase of Enterobacter cloacae P99

^{□}(Column 10): calculated using the penicillin detector of the Beta-Star® Test

TABLE 1
Characteristics of the carbenicillin, oxacillin and benzylpenicillin conjugates

| | Mass | IR (cm⁻¹) | | | | | | | Kₐ [symbol] × | |
|---|---|---|---|---|---|---|---|---|---|---|
| Conjugate | MP | v₁ | v₂ | v₃ | v₄ | v₅ | v₆ | Kᵢ^{x} nM | 10⁻⁶ L.M⁻¹ | Structure formula |
| 1 | 635*⁺ | 1765 | 1740 | 1725 | 1682 | | | 70 ± 43 | | 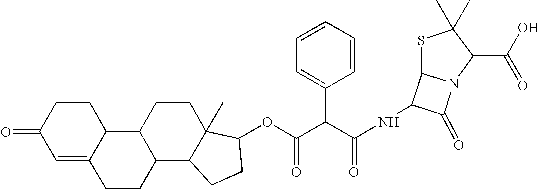 |
| 2 | 704^{#-} | 1759 | 1741 | 1710 | 1701 | 1679 | 1640 | | 4.6 ± 3.0 | 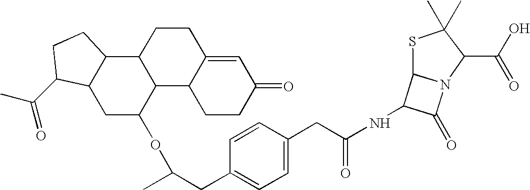 |
| 3 | 694*⁺ | 1764 | 1752 | 1721 | 1673 | | | 84 ± 47 | | 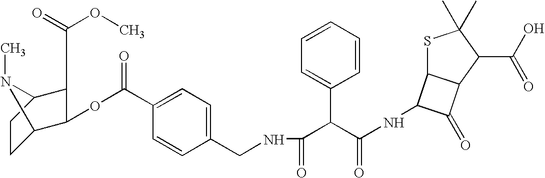 |
| 4 | 699*⁺ | 3294 | 1776 | 1716 | 1654 | | | 24 ± 26 | | 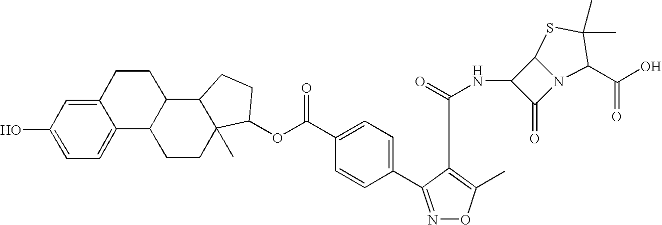 |
| 5 | 699*⁻ | 1767 | 1726 | 1717 | 1677 | 1664 | | 18 ± 23 | | 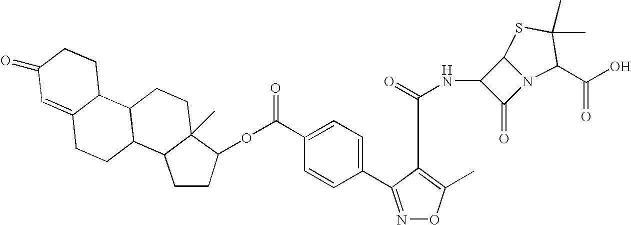 |
| 6 | 714*⁻ | 1788 | 1717 | 1674 | 1638 | 1279 | | 22 ± 19 | | 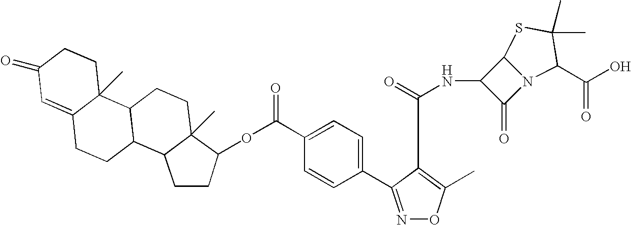 |
| 7 | 759*⁺ | 1767 | 1744 | 1722 | 1699 | 1674 | | 19 ± 27 | | 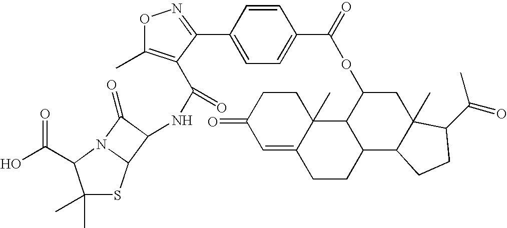 |
| 8 | 802*⁺ | 1781 | 1746 | 1719 | 1673 | | | 23 ± 18 | | 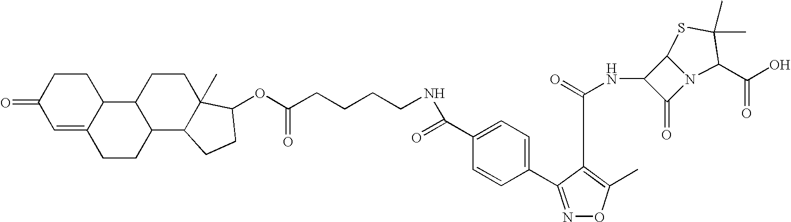 |
| 9 | 816*⁺ | 1766 | 1738 | 1724 | 1680 | 1662 | | 17 ± 21 | | 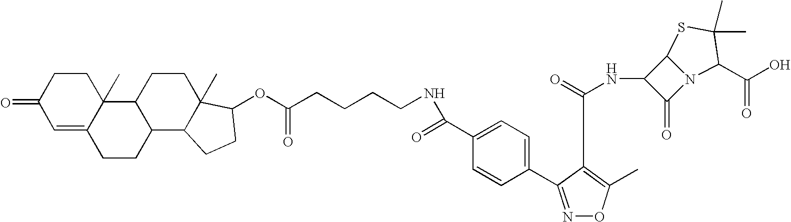 |
| 10 | 751*⁺ | 3302 | 1764 | 1726 | 1668 | | | 14 ± 17 | | 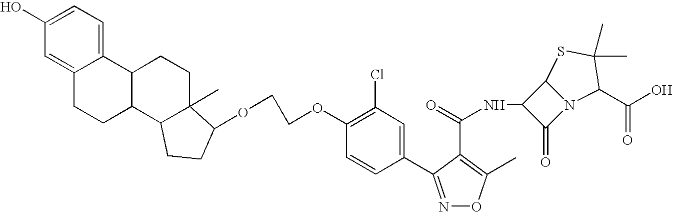 |
| 11 | 753*⁺ | 1763 | 1725 | 1683 | 1668 | | | 12 ± 14 | | 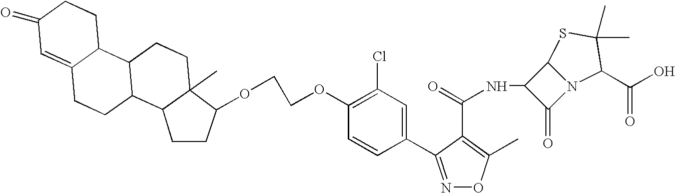 |
| 12 | 767*⁺ | 1786 | 1718 | 1675 | 1641 | | | 15 ± 16 | | 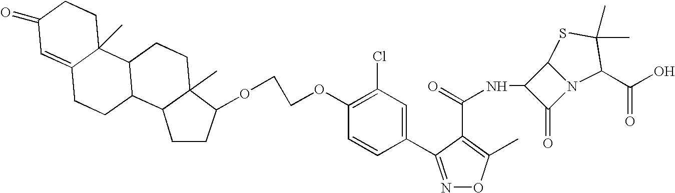 |
| 13 | 809*⁺ | 1765 | 1723 | 1702 | 1668 | | | 20 ± 21 | | 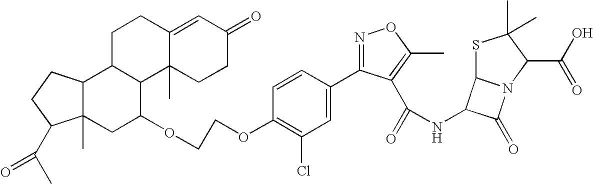 |
| 14 | 758*⁺ | 3299 | 1786 | 1717 | 1672 | | | 18 ± 23 | | 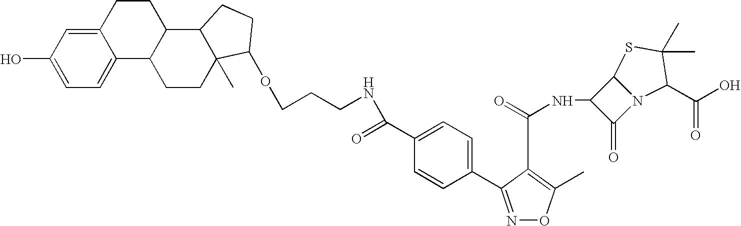 |
| 15 | 760*⁺ | 1783 | 1720 | 1677 | | | | 28 ± 22 | | 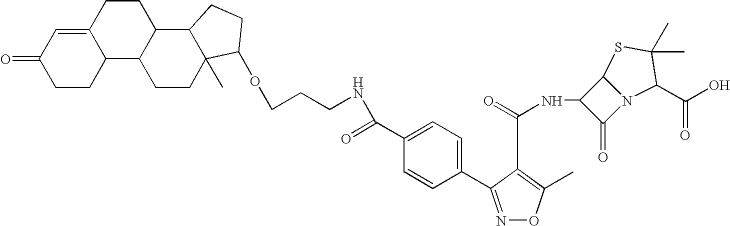 |
| 16 | 774*⁺ | 1769 | 1726 | | | | | 26 ± 27 | | 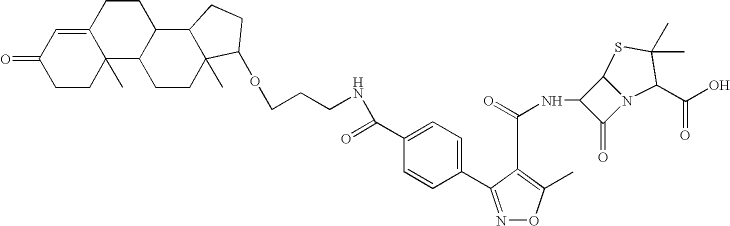 |
| 17 | 816*⁺ | 1764 | 1726 | 1711 | 1670 | | | 21 ± 24 | | 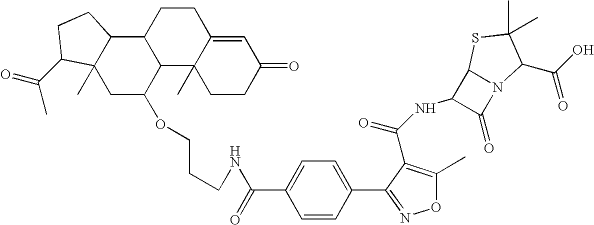 |
| 18 | 788*⁺ | 1784 | 1739 | 1720 | 1668 | | | 23 ± 19 | | 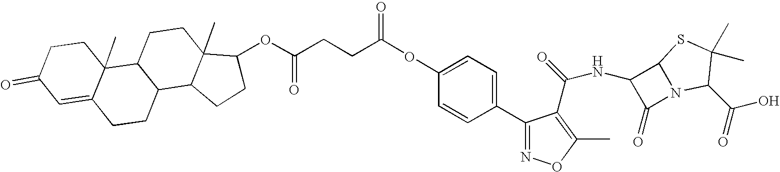 |
| 19 | 774*⁺ | 1785 | 1742 | 1718 | 1671 | | | 21 ± 24 | | 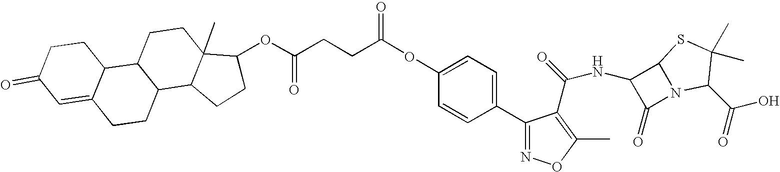 |
| 20 | 760*⁺ | 1787 | 1718 | 1680 | | | | 29 ± 27 | | 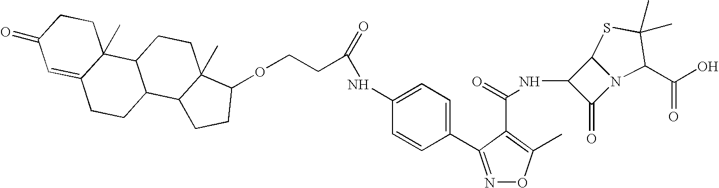 |
| 21 | 746*⁺ | 1784 | 1717 | 1678 | | | | 30 ± 33 | | 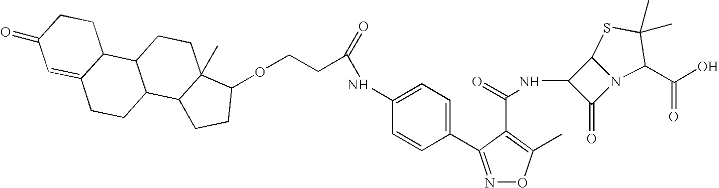 |
| 22 | 802*⁺ | 1763 | 1722 | 1703 | 1670 | | | 25 ± 23 | | 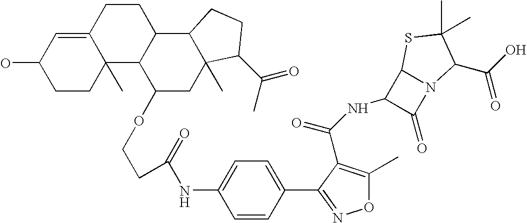 |
| 23 | 744*⁺ | 3307 | 1721 | 1667 | | | | 19 ± 21 | | 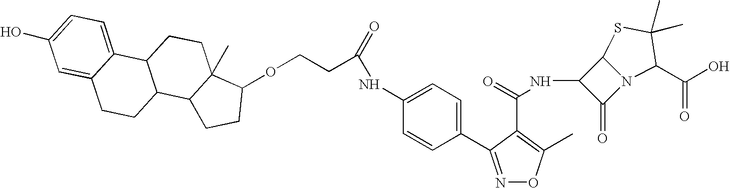 |
| 24 | 761*⁺ | 1768 | 1750 | 1722 | 1676 | | | 21 ± 26 | | 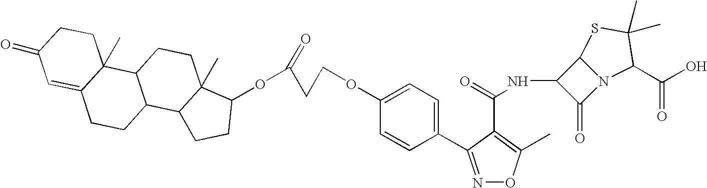 |
| 25 | 747*⁺ | 1766 | 1742 | 1724 | 1683 | 1674 | | 28 ± 31 | | 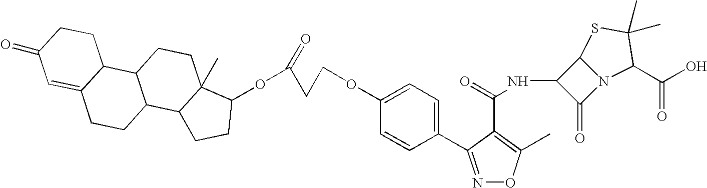 |
| 26 | 846*⁺ | 1764 | 1745 | 1724 | 1671 | | | 29 ± 26 | | 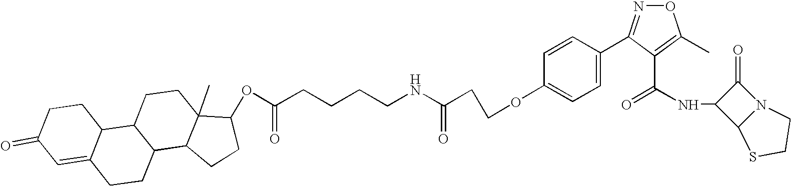 |
| 27 | 860*⁺ | 1766 | 1740 | 1725 | 1669 | | | 23 ± 25 | | 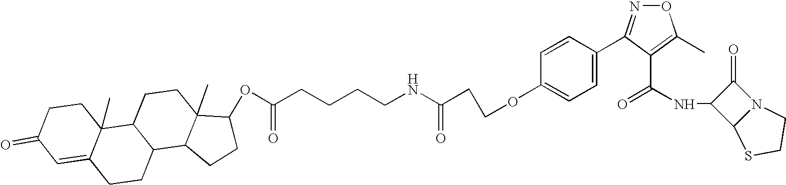 |
| 28 | 803*⁺ | 1765 | 1741 | 1726 | 1710 | 1674 | | 26 ± 26 | | 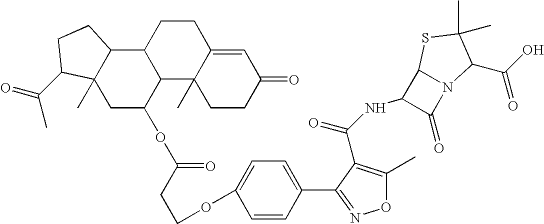 |
| 29 | 745*⁺ | 3306 | 1765 | 1743 | 1724 | 1677 | | 21 ± 24 | | 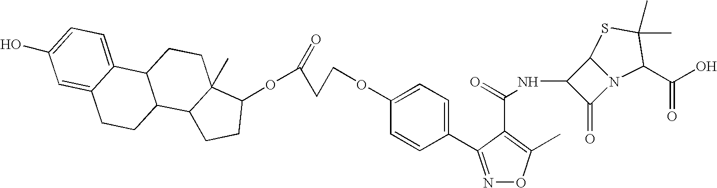 |
| 30 | 648^{#-} | 1760 | 1737 | 1700 | 1681 | 1649 | | | 3.8 ± 2.9 | 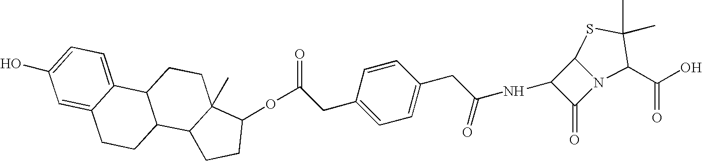 |
| 31 | 662^{#-} | 1764 | 1740 | 1703 | 1678 | 1643 | | | 4.1 ± 3.2 | 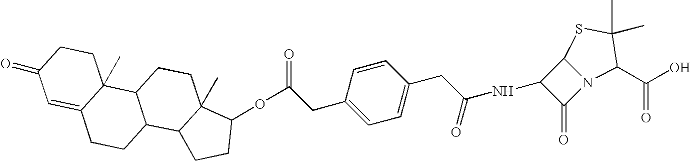 |
| 32 | 646^{#-} | 3297 | 1766 | 1741 | 1703 | 1646 | | | 4.8 ± 3.5 | 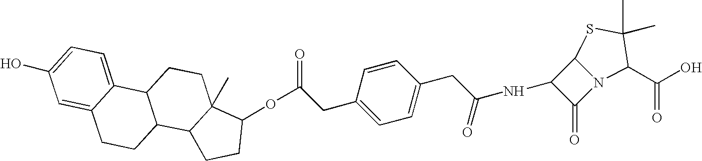 |

### EXAMPLE 1

### Based on β-Lactamase Detection with Nitrocefin

1. Choice of the Enzyme and its Substrate

The enzyme showing the best couple of kinetic parameters for the nandrolone carbenicillinate conjugate and the reporter substrate nitrocefin is the type C β-lactamase from Enterobacter cloacae P99. The Kₘ obtained respectively for the conjugate and for the reporter substrate are 74 nM and 25 μM while the k_{cat} determined respectively for the same compounds are 0.25 s⁻¹ and 780 s⁻¹.

2. Assay of Nandrolone (Table 2)

The assay of nandrolone, using the conjugate 1 as inhibitor, was carried out in the following manner: a nandrolone sample is added to a solution of nandrolone carbenicillinate. The mixture thus obtained is supplemented with dilute antibody serum and then vortexed for 30 seconds. It is then supplemented with a solution of reporter substrate, buffer and enzyme. After brief stirring, the absorbance of the medium at 482 nm is continuously read for 5 minutes and the hydrolysis rate of nitrocefin is determined in the absence of antibody (Vᵢ), in the presence of antibody (V_{ab}) and in the presence of various quantities of nandrolone (V_{dos}). From these data, it appears that the minimum concentration of nandrolone detectable in the measurement medium is of the order of 0.02 μM, which corresponds to 10 picomoles of substance (measurement volume=500 μl).

TABLE 2

| S | E | I | Serum | [steroid] | V_{dos} | V_{ab} | Vᵢ |
|---|---|---|---|---|---|---|---|
| μM | pM | nM | dilution | nM | nM · s⁻¹ | nM · s⁻¹ | nM · s⁻¹ |
| 60 | 250 | 240 | 625 | 20 | 186 | 190 | 116 |
| 60 | 250 | 240 | 625 | 80 | 174 | 190 | 116 |
| 60 | 250 | 240 | 625 | 160 | 161 | 190 | 116 |
| 60 | 250 | 240 | 625 | 200 | 152 | 190 | 116 |

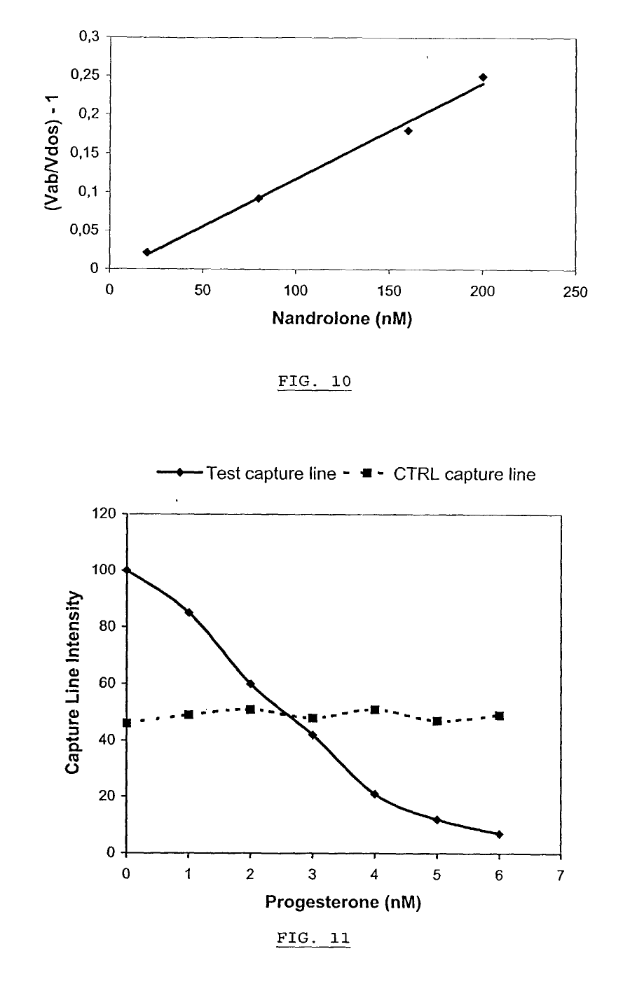

FIG. 10, which describes the variation of [(V_{ab}/V_{dos})−1] as a function of the quantity of nandrolone, shows a linear relationship between the two parameters.

### EXAMPLE 2

### Based on β-Lactamase Detection with Cephalexin and the Iodine-Starch System

1. Choice of the Enzyme and its Substrate

The enzyme showing the best couple of kinetic parameters for the nandrolone carbenicillinate conjugate and the reporter substrate cephalexin is the type C β-lactamase from Enterobacter cloacae P99. The Kₘ obtained respectively for the conjugate and for the reporter substrate are 74 nM and 10 μM while the k_{cat} determined respectively for the same compounds are 0.25 s⁻¹ and 100 s⁻¹.

2. Assay of Nandrolone (Table 3)

The assay of nandrolone, using the conjugate 1 as inhibitor, was carried out in the following manner: a nandrolone sample is added to a solution of nandrolone carbenicillinate. The mixture thus obtained is supplemented with dilute antibody serum and then vortexed for 30 seconds. It is then supplemented with a solution of reporter substrate, buffer and enzyme. The variation in color is observed at 620 nm which corresponds to an iodine consumption of the cephalexin degraded during the measuring process. The iodine is generated in the following manner: 100 μl of a mixed solution of starch cadmium iodide and of potassium iodate (0.002N CdI₂−0.0024N KIO₃−1.5% starch) are taken and supplemented with 100 μl of a 0.1N solution of sodium salt of DTPA before the pH is brought to 2 by addition of 1M HCl; after reaction, the medium is brought to pH 7 by addition of a sufficient quantity of 0.02M Hepes buffer and this solution is introduced into the immunoassay reagents. From the data given at Table 8, it appears that the minimum concentration of nandrolone detectable in the measurement medium is of the order of 0.01 μM, which corresponds to 5 picomoles of substance (measurement volume=500 μl).

TABLE 3

| S | E | I | Serum | [steroid] | V_{dos} | V_{ab} | Vᵢ |
|---|---|---|---|---|---|---|---|
| μM | nM | nM | dilution | nM | nM · s⁻¹ | nM · s⁻¹ | nM · s⁻¹ |
| 20 | 1 | 150 | 1000 | 12.5 | 43 | 45 | 28.5 |
| 20 | 1 | 150 | 1000 | 50 | 36.3 | 45 | 28.5 |
| 20 | 1 | 150 | 1000 | 100 | 30.9 | 45 | 28.5 |
| 20 | 1 | 150 | 1000 | 125 | 28.4 | 45 | 28.5 |

### EXAMPLE 3

### Based on β-Lactamase Detection with Nitrocefin

1. Choice of the Enzyme and its Substrate

The enzyme showing the best couple of kinetic parameters for the progesterone oxacillinate conjugate and the reporter substrate nitrocefin is the type C β-lactamase from Enterobacter cloacae P99. The Kₘ obtained respectively for the conjugate and for the reporter substrate are 1.7 nM and 25 μM while the k_{cat} determined respectively for the same compounds are 0.01 s⁻¹: and 780 s⁻¹.

2. Assay of Progesterone (Table 4)

The assay of progesterone, using the conjugate 7 as inhibitor, was carried out in the following manner: a progesterone sample is added to a solution of progesterone oxacillinate. The mixture thus obtained is supplemented with dilute antibody serum and then vortexed for 30 seconds. It is then supplemented with a solution of reporter substrate, buffer and enzyme. After brief stirring, the absorbance of the medium at 482 nm is continuously read for 10 minutes and the hydrolysis rate of nitrocefin at steady state (after approximately 4 minutes) is determined in the absence of antibody (Vᵢ), in the presence of antibody (V_{b}) and in the presence of various quantities of progesterone (V_{dos}). From these data, it appears that the minimum concentration of progesterone detectable in the measurement medium is of the order of 2 nM which corresponds to 1 picomole of substance (measurement volume=500 μl).

TABLE 4

| | | | | [ster- | | | |
|---|---|---|---|---|---|---|---|
| S | E | I | Serum | oid] | V_{dos} | V_{ab} | Vᵢ |
| μM | pM | nM | dilution | nM | nM · s⁻¹ | nM · s⁻¹ | nM · s⁻¹ |
| 100 | 160 | 10 | 1250 | 2 | 94 | 96 | 52 |
| 100 | 160 | 10 | 1250 | 4 | 92 | 96 | 52 |
| 100 | 160 | 10 | 1250 | 8 | 89 | 96 | 52 |
| 100 | 160 | 10 | 1250 | 16 | 86 | 96 | 52 |
| 100 | 160 | 10 | 1250 | 24 | 82 | 96 | 52 |
| 100 | 160 | 10 | 1250 | 32 | 77 | 96 | 52 |

### EXAMPLE 4

### Based on Beta-Star® Detection

1. Choice of the Detector and the Conjugate

The detector showing the best affinity for the progesterone benzylpenicillinate conjugate is the polypeptide included in the Beta-star® kit. This genetic engineering peptide has kept a very high affinity for benzylpenicillin but has no enzymatic activity. It is thus coupled to colored particles for allowing sensitive detection.

2. Assay of Progesterone

The assay of progesterone, using the conjugate 2 as inhibitor, was carried out in the following manner: a progesterone sample is added to a solution of 4 nM progesterone benzylpenicillinate. The mixture thus obtained is supplemented with dilute antibody serum and buffer to obtain 0.5 ml and then vortexed for 30 seconds. This solution is transferred into an individual receptor vial. After swirling the vial in order to dissolve the solid material, the solution is incubated for 5 minutes at 47° C. and allowed to migrate into a dipstick for a further period of 5 minutes at the same temperature. After reaction, two red lines appear: the first one (CTRL capture line) is a reference while the intensity of the second one (test capture line), after reading with an appropriate strip reader, can be correlated to concentration of the sample. The minimum concentration of progesterone detectable in the measurement medium is of the order of 1 nM, which corresponds to 500 femtomoles of substance (measurement volume=500 μl).

TABLE 5

| Progesterone | Test capture line | CTRL capture line |
|---|---|---|
| nM | Relative intensity | Relative intensity |
| 0 | 100 | 46 |
| 1 | 85 | 49 |
| 2 | 60 | 51 |
| 3 | 42 | 48 |
| 4 | 21 | 51 |
| 5 | 12 | 47 |
| 6 | 7 | 49 |

FIG. 11, which describes the variation of the red color intensity scanned on the dipstick as a function of the quantity of progesterone, shows a sigmoid relationship between the two parameters.

### Examples of Protecting Agents

1 Interference of the Serum on the β-Lactamase Based Systems

Serum increases the spontaneous hydrolysis rate of nitrocefin but decreases the catalytic activity of the enzyme. From data given at Table 6, it can be observed that, in the presence of 0.1% sodium azide, the spontaneous hydrolysis of nitrocefin is highly reduced and that a further addition of phenylbutazone at a concentration of 70 mg/l can restore the catalytic activity of the enzyme. Examples of assay of nandrolone in the presence of these two protecting agents are given in this table.

TABLE 6

| Vₘₑₛ | V | Q_{enz} | Q_{sub} | Qᵢₙₕ | Qₐₙₐₗ | DS | Serum | | | |
|---|---|---|---|---|---|---|---|---|---|---|
| μl | μM/s | nM | nM | nM | nM | A | % | Treatment | Buffer | pH |
| 500 | 0.219 | 0.25 | 75 | — | — | — | 0 | — | hepes | 8.2 |
| 500 | 0.143 | 0.25 | 75 | 240 | — | — | 0 | — | hepes | 8.2 |
| 500 | 0.206 | 0.25 | 75 | 240 | — | 625 | 0 | — | hepes | 8.2 |
| 500 | 0.14 | — | 75 | — | — | — | 24 | — | hepes | 8.2 |
| 500 | 0.05 | — | 75 | — | — | — | 2 | — | hepes | 8.2 |
| 500 | 0.203 | 0.25 | 75 | — | — | — | 2 | — | hepes | 8.2 |
| 500 | 0.156 | 0.25 | 75 | 240 | — | — | 2 | — | hepes | 8.2 |
| 500 | 0.215 | 0.25 | 75 | 240 | — | 625 | 2 | — | hepes | 8.2 |
| 500 | 0.220 | 0.25 | 75 | — | — | — | 0 | — | hepes | 8.2 |
| 500 | 0.110 | 0.25 | 75 | 300 | — | — | 0 | — | hepes | 8.2 |
| 500 | 0.197 | 0.25 | 75 | 300 | — | 500 | 0 | — | hepes | 8.2 |
| 500 | 0.196 | 0.25 | 75 | 300 | — | 454 | 0 | — | hepes | 8.2 |
| 500 | 0.188 | 0.25 | 75 | 300 | — | 556 | 0 | — | hepes | 8.2 |
| 500 | 0.148 | 0.25 | 75 | 300 | — | 500 | 2 | — | hepes | 8.2 |
| 500 | 0.134 | 0.25 | 75 | 300 | 110 | 500 | 2 | — | hepes | 8.2 |
| 500 | 0.127 | 0.25 | 75 | 300 | 400 | 500 | 2 | — | hepes | 8.2 |
| 500 | 0.111 | 0.25 | 75 | 300 | 2000 | 500 | 2 | — | hepes | 8.2 |
| 500 | 0.015 | — | 75 | — | — | — | 2 | NaN₃ 0.1% | mixed | 7 |
| 500 | 0.101 | 0.25 | 75 | 300 | — | — | 2 | NaN₃ 0.1% | mixed | 7 |
| 500 | 0.193 | 0.25 | 75 | — | — | — | 2 | NaN₃ 0.1% | mixed | 7 |
| 500 | 0.192 | 0.25 | 75 | 300 | — | 500 | 2 | NaN₃ 0.1% | mixed | 7 |
| 500 | 0.170 | 0.25 | 75 | 300 | 22 | 500 | 2 | NaN₃ 0.1% | mixed | 7 |
| 500 | 0.165 | 0.25 | 75 | 300 | 110 | 500 | 2 | NaN₃ 0.1% | mixed | 7 |
| 500 | 01.35 | 0.25 | 75 | 300 | 400 | 500 | 2 | NaN₃ 0.1% | mixed | 7 |
| 500 | 0.125 | 0.25 | 75 | 300 | 2000 | 500 | 2 | NaN₃ 0.1% | mixed | 7 |
| 450 | 0.12 | — | 75 | — | — | — | 10 | — | citrate | 7 |
| 450 | 0.022 | — | 75 | — | — | — | 10 | Butazolidine | citrate | 7 |
| 450 | 0.276 | 0.75 | 75 | — | — | — | 10 | Butazolidine | citrate | 7 |
| 450 | 0.246 | 0.75 | 75 | 275 | — | — | 10 | Butazolidine | citrate | 7 |
| 450 | 0.215 | 0.75 | 75 | 275 | 11 | 500 | 10 | Butazolidine | citrate | 7 |
| 450 | 0.207 | 0.75 | 75 | 275 | 22 | 500 | 10 | Butazolidine | citrate | 7 |
| 450 | 0.184 | 0.75 | 75 | 275 | 110 | 500 | 10 | Butazolidine | citrate | 7 |

legend
Vₘₑₛ: Final volume in which the enzymatic kinetics is monitored.
V: Rate of degradation of the substrate (nitrocefin).
Q_{enz}: Concentration of P99 β-lactamase in the final volume.
Q_{sub—}: Concentration of nitrocefin in the final volume.
Qᵢₙₕ: Concentration of inhibitor in the final volume.
Q_{anal—}: Concentration of analyte in the final volume.
DSA: Dilution of the serum containing the antinandrolone antibodies.
Serum: Percentage of serum in the final volume.
Treatment: Protecting agent tested (Butazolidine = phenylbutazone 70 mg/l).
Buffer: Buffer used during the measurements (hepes = hepes 2.5 mM; PBS = 0.1 M phosphate + 0.15 M NaCl; mixed = 9/1 mixture of PBS and of Hepes; citrate = citrate 0.005 M).
pH: pH of the buffer used during the measurements.

2. Interference of antibiotics

Most of the classes of antibiotics (macrolides, quinolones, etc.) have no effects on the assay. Nevertheless, β-lactamic antibiotics inhibit the enzymatic activities or the recognition by the penicillin detectors. Fortunately, β-lactamic antibiotics are well hydrolysed by β-lactamases and the catalytic activity of type B β-lactamases having zinc atom in their catalytic site can be modulated by complexing agents as ethylene diamino tertaacetic acid (EDTA).

The sample containing the interfering antibiotic is incubated for 30 seconds with 50 μl of a solution of the β-lactamase of Bacillus cereus (1 μg/ml), then supplemented with 25 μl 0.02 M EDTA and further incubated for 15 seconds. The assay is continued following the general procedure. This procedure, applied to samples supplemented with astreonam or cephalexin, allowed to restore more than 95% of the native enzymatic activity.

Example of Cephalexin Determination by the Iodine-Starch System

1. Preparation of the Stock Solutions

100 ml of a mixed solution of 0.003 N cadmium iodide and 0.00275 N potassium iodate are prepared by dissolving, respectively, 54.92 mg of cadmium iodide and 9.77 mg of potassium iodate in a 10 mM hepes buffer solution pH 8.2 charged with 1% starch paste. 100 ml of 0.2 M DTPA solution in hepes buffer are also prepared. Finally, a 1 mM stock solution of cephalexin is prepared by dissolving 34 mg of cephalexin in 100 ml of the hepes buffer described above.

2. Preparation of the Scale

The stock solution of cephalexin is hydrolysed and diluted by collecting 1 ml which is treated with 4 ml of 0.001 M sodium hydroxide for 15 minutes, and then adjusted to 100 ml with the hepes buffer.

A starch iodine solution is prepared by collecting 20 ml of mixed solution, 25 ml of DTPA and 3 ml of glacial acetic acid, and then the volume is adjusted with hepes buffer (starch iodine solution).

A hydrolysed cephalexin concentration scale from 1 to 6 μM is prepared by removing volumes of 100 to 600 μl to which 100 μl of the starch iodine solution are added. The volume is adjusted to 1 ml with hepes buffer.

The final absorbance of the solutions is measured after 5 minutes at 620 nm.

TABLE 7

| Concentration | |
|---|---|
| (μM) | Absorbance |
| 0 | 0.963 |
| 1 | 0.831 |
| 2 | 0.682 |
| 3 | 0.549 |
| 4 | 0.428 |
| 5 | 0.280 |
| 6 | 0.155 |

From the data of Table 7, a correlation coefficient of 0.9961 is obtained as well as a linear relation corresponding to the equation: Y=−0.1345X+0.96.

3. Assay

The cephalexin stock solution is diluted so as to obtain a concentration of between 0.5 and 2 mM.

The preceding solution is hydrolysed and diluted according to the technique used for the preparation of the scale; then, 250 μl of this solution are collected, supplemented with 100 μl of starch iodine solution and the volume is adjusted to 1 ml with hepes buffer.

After 5 minutes, the absorbance is read at 620 nm and the concentration is calculated relative to the pre-established scale.

1. A conjugate of a hapten to a natural or synthetic β-lactam derivative, wherein the β-lactam derivative is at least partially constitutive of the conjugating arm.

2. A conjugate according to claim 1, wherein said conjugating arm is bound to the amine group located on the β-lactam nucleus.

3. A conjugate according to claim 2, wherein the part of the conjugating arm located between the hapten moiety and the side chain of the β-lactam derivative includes a number of atoms comprised between 0 and 10.

4. A conjugate according to claim 3, wherein the number of atoms is comprised between 0 and 4.

5. A conjugate according to claim 3, wherein said atoms are carbon atoms and heteroatoms selected from the group consisting of oxygen, sulphur and nitrogen.

6. A conjugate according to claim 1, wherein the hapten is selected from the group consisting of steroids, drugs, drugs of abuse and medicines.

7. A conjugate according to claim 1, wherein said β-lactam derivative is selected from the group consisting of penicillins and cephalosporins.

8. A conjugate according to claim 6 wherein the hapten is chosen from the group consisting of nandrolone, testosterone, progesterone, estradiol and cocaine.

9. A conjugate according to claim 7, wherein the β-lactam derivative is selected from the group consisting of carbenicillin, oxacillin, cefuroxime, cefotaxime, methicillin, benzylpenicillin and phenoxymethylpenicillin.

10. A conjugate according to claim 8 or 9 wherein said conjugate is selected from the group consisting of nandrolone carbenicillinate, cocaine carbenicillinate, progesterone oxacillinate and progesterone benzylpenicillinate.

11. Method for the immunoassay of a hapten involving a β-lactam derivative-based inhibitor-hapten conjugate according to claim 1, wherein:
said β-lactam derivative-based inhibitor-hapten conjugate binds, competitively with the free hapten to be assayed in a solution, to an anti-hapten antibody
said conjugate, when unbound to said antibody, is capable to bind to a protein receptor site for the β-lactam moiety, the antibody-bound conjugate being unable to bind to said receptor, owing to steric hindrance
the recognition of the conjugate binding to the receptor is associated to detection and/or quantification means.

12. Method according to claim 11, wherein the recognition of the conjugate binding to the protein receptor is related to the modulation of an enzyme activity of said protein against a reporter substrate.

13. Method for the immunoassay in an homogeneous phase of a hapten involving a β-lactam derivative-based inhibitor-hapten conjugate according to claim 11, comprising the steps of:
adding a known quantity of inhibitor-hapten conjugate (IH_{b}) to a sample solution containing the free hapten (H_{f}) to be detected and/or quantified;
adding a quantity of antibody (AB) directed at the hapten in its free and conjugate state and related to the quantity of said conjugate in solution;
adding to the solution a β-lactamase having an active site for two substrates entering into competition on said active site, the first substrate being a reporter substrate (S) transforming into a product detectable and/or quantifiable, the second substrate being the inhibitor-hapten conjugate (IH_{b}) modulating the rate of hydrolysis of the reporter substrate (S), the higher the quantity of free hapten (H_{f}) initially in solution, the lower the quantity of inhibitor-hapten conjugate having bound one said antibody (IH_{b}-AB) and the higher the quantity of said conjugate binding at the active site (IH_{b}-E) and the lower the enzymatic activity against the reporter substrate (S).

14. Method according to claim 13, wherein the β-lactamase is a class C β-lactamase.

15. Method according to claim 14, wherein the class C β-lactamase is selected from the group consisiting of class C β-lactamases extracted from Enterobacter cloacae, Escherichia coli and Citrobacter frundii.

16. Method for the immunoassay of a hapten involving a β-lactam derivative-based inhibitor-hapten conjugate according to claim 11, comprising the steps of:
adding a known quantity of inhibitor-hapten conjugate (IH_{b}) to a sample solution containing the free hapten (H) to be detected and/or quantified;
adding a quantity of antibody (AB) directed at the hapten in its free and conjugate state and corresponding to the quantity of said conjugate in solution;
adding to the solution a penicillin detector (D) capable of specific recognition of the β-lactamic moiety of said conjugate (IH_{b}), the bound quantity of said conjugate (IH_{b}) to said detector (D) being modulated in a competitive reaction of the free hapten (H_{f}) initially in solution and said conjugate (IH_{b}) with said antibody (AB).

17. Method according to claim 16, wherein the penicillin detector is a polypeptide included in a detection kit selected from the group consisting of BetaSTAR®, SNA® P Beta Lactam, Penzym®, Parallux® Beta Lactam assay, Charm Farm Test®, Delvo-X-press®, Delvotest® P and Delvo® test SP.

18. Method according to claim 12, wherein the reporter substrate is nitrocefin.

19. Method according to claim 12, wherein the reporter substrate is cephalexin.

20. Method according to claim 11 or 16, wherein substances are added to the hapten-containing solution to be assayed for removing possible interference.

21. Method according to claim 20, wherein said substances are selected from the group consisting of agents for protecting β-lactamase and penicillin detector, agents for protecting reporter substrate, agents for protecting hapten-inhibitor complex and decontaminating agents.

22. Method according to claim 21, wherein said substances are selected from the group consisting of class B β-lactamases, sodium azide and phenylbutazone.

23. Method according to claim 22, wherein the class B β-lactamase is extracted from Bacillus cereus.

24. Method according to claim 12, wherein a starch-iodine color system is used for revealing the hydrolysis rate of the reporter substrate, iodine being generated in a solution of starch paste stabilized by addition of cadmium iodide.

25. Method according to claim 24, wherein the iodine is generated from cadmium iodide in the presence of a complexing agent and an oxidizing agent in a medium of appropriate pH and then bringing the revealing reagent to the working pH.

26. Method according to claim 25, wherein the complexing agent used is DTPA.

27. Method according to claim 25, wherein the oxidizing agent is an iodate or a periodate.

28. A kit of reagents for the immunoassay of a hapten, comprising:
a buffer liquid for immunoassay containing a known quantity of β-lactam derivative-based inhibitor-hapten conjugate, said β-lactam derivative being selected from the group consisting of carbenicillin, oxacillin, cefuroxime, cefotaxime, methicillin, benzylpenicillin and phenoxymethylpenicillin;
a quantity of antibody directed at the hapten in its free and conjugate state and related to the quantity of inhibitor-hapten conjugate in solution;
a β-lactamase enzyme associated with a known quantity of reporter substrate to be hydrolyzed by the enzyme or a penicillin detector capable of specific recognition of the β-lactamic moiety of said conjugate;
reagents for detecting and/or quantifying enzyme or penicillin detector activity.

29. A kit of reagents according to claim 28, comprising at least one reagent selected from the group consisting of agents for protecting β-lactamase, agents for protecting reporter substrate, agents for protecting hapten-inhibitor complex and decontaminating agents.
